(19) United States
(12) Patent Application Publication
(10) Pub. No.: US 2023/0083927 A1
(43) Pub. Date: Mar. 16, 2023

(54) ELECTRONIC VAPOR LIQUID COMPOSITION AND METHOD OF USE

(71) Applicant: NOAH GRAY, Arvada, CO (US)

(72) Inventor: NOAH GRAY, Arvada, CO (US)

(73) Assignee: Sokeconoma Technologies LLC, New York, NY (US)

(21) Appl. No.: 17/471,475

(22) Filed: Sep. 10, 2021

(51) Int. Cl. A61K 47/10; A61K 47/44; A61K 9/00; A61K 45/06; A24B 15/167

(52) U.S. Cl. CPC A61K 47/10; A61K 47/44; A61K 9/007; A61K 45/06; A24B 15/167

(57) ABSTRACT

The invention relates to a vaping compound which is comprised of a vaporizing base which is formed from the following ingredients propylene glycol, MCT oil and vegetable glycerin, or combination of the oils, flavoring, water and one or more active ingredients.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### Field of the Invention

The invention relates to the field of e-liquid for use in electronic cigarettes which utilizes a vaporizing base consisting of propylene glycol, vegetable glycerin, MCT Oil, terpenes, water, or any mixture thereof, mixed with an herbal powder extract, liquid or oleoresin.

### DESCRIPTION OF RELATED ART

Currently the state of the art includes a number of E-cigarettes, and e-cigarette liquid which are very popular alternatives to utilizing cigarettes and other tobacco products. The present invention, and inventive system, is a new and novel electronic cigarette liquid used in conjunction with electronic cigarettes. The present invention can be used for medicinal, recreational, or alternative purposes desired by the user of the product. The present invention is designed to give an e-liquid user an alternative compound than existing nicotine based liquids.

In particular there is a need for an e-cigarette liquid that provides alternative compounds such as medicinal ingredients.

### BRIEF SUMMARY OF THE INVENTION

The instant invention in one form is directed to a new and easy to use compound for electronic cigarette liquid used in conjunction with electronic cigarettes. The present invention can be used to enhance effects for relaxation, medicinal, recreational, or alternative purposes desired by a user of an e-cigarette. The present invention provides the following benefits to other e-liquids on the market place by using extracts or herbal substances which are compounded with a e-liquid carrier for use in an e-liquid in an e-cigarette. The extracts come from select herbal plants and extracts used in Native American and Eastern cultures along with other chemicals which are capable of providing relaxing or medicinal effects to the user of the e-cigarette liquid.

An advantage of the present invention is the ability of the instant invention to provide the user of an e-cigarette an e-liquid synthesized using a carrier base of propylene glycol, vegetable glycerin, MCT Oil, terpenes, water, or any mixture thereof, mixed with an herbal powder extract (or synthetically derived active ingredients) which is generally at a ratio of 0.001 g-5.0 g per ml. The herbal extract/synthetically derived active ingredients can be any of the following: Kanna (Sceletium tortuosum), Salvia (Salvia divinorum), Salvia eivinorm, Kratom (Mitragyna speciosa), Celandine poppy (Stylophorum diphyllum), Mugwort (Artemisia), Coltsfoot leaf (Tussilago farfara), California poppy (Eschscholzia californica), Sinicuichi (Heimia salicifolia), St. John's Wort (Hypericum perforatum), Yerba lenna yesca (Artemisia scoparia), Calea zacatechichi (Calea ternifolia), Blue lotus (Nymphaea caerulea), Leonurus sibericus (Leonurus sibiricus), Wild dagga (Leonotis leonurus), Valarien (Valeriana officinalis), Klip dagga (Leonotis nepetifolia), Damiana (Turnera diffusa), Kava (Piper methysticum), Scotch broom tops (Cytisus scoparius), Indian warrior (Pedicularis densiflora), Wild lettuce (Lactuca virosa), Skullcap (Scutellaria lateriflora), Red Clover (Trifolium pretense), Psilocybin Mushroom (Psilocybin Cubensis), Psilocin Mushroom (Copelandia, Gymnopilus, Inocybe, Panaeolus, Pholiotina, Pluteus, and Psilocybe), Lions Mane (Hericium Erinaceus), Cordyceps (Cordyceps Sinesis), Reishi Mushroom (Ganoderma Lingzhi), Chaga Mushroom (Inonotus Obliquus), Cacao (Forastero, Criollo, Trinitario), mimosa shrub (mimosa hostilis), Hemp/cannabis and/or combinations therein.

The instant invention overcomes the tendency for water molecules to attract each other and oil molecules to stick together by utilization of high shear mixing. That causes oil and water to form two separate layers. Water molecules pack closer together, so they sink to the bottom, leaving oil sitting on top of the water. The high shear mixing causes the oil to break up into tiny droplets and becomes distributed in the water forming a mixture.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 illustrates a chart of the various plants from which the herbal additives of the present invention can be derived.

FIG. 2 illustrates a flow chart for creating one embodiment of the present invention for Mixture of the instant invention.

FIG. 3 illustrates a flow chart for creating second embodiment of the present invention for Mixture of the instant invention.

FIG. 4 illustrates a flow chart for creating third embodiment of the present invention for Mixture of the instant invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms e-liquid, compound, solution and liquid are used interchangeably to mean a liquid that is formulated using the process of the instant invention and is capable of being used in an e-cigarette for the purpose of vapor production.

The terms e-cigarette and smoking device are used interchangeably to mean an e-cigarette that is capable of using a e-liquid formulated using the process of the instant invention.

The term inhalers as used in the specification can be either an e-cigarette, metered dose inhalers, pressurized metered dose inhalers (MDIs), Breath-activated inhalers or inhalers with spacer devices or nebulizers.

The term emulsification or emulsion as used in this specification is a mixture of two or more liquids that are normally immiscible owing to liquid-liquid phase separation. Emulsions are part of a more general class of two-phase systems of matter called colloids.

Prior to a discussion of the preferred embodiment of the invention, it should be understood that while the features and advantages of the invention are illustrated in terms of an e-liquid, the invention can be used with any type of device that is capable of aspirating, vaporizing or misting a liquid for human inhalation such as inhalable insulin delivery devices, nebulizer, e-cigarette or metered dose inhalers.

The instant invention vaping compound or e-liquid comprises the following components:
a. vaporizing base is formulated from propylene glycol, vegetable glycerin, MCT oil, terpenes, flavorings, water, or any mixture thereof;
b. an herbal powder extract (or synthetically derived) active ingredients;

More specifically the instant invention is a vaping compound or e-liquid that is comprises of the following ingredients but not limited to:
a. propylene glycol, MCT oil and vegetable glycerin, terpenes or combination of the oils; and
b. water; and
c. active ingredients.

The active ingredients which comprise of herbal powder extract, liquid, oleoresin or synthetically derived active ingredients are incorporated into the vaporizing base such that the ratio of herbal powder extract or synthetically derived active ingredients to vaporizing base is generally at a 0.001 g-5.0 g per mL of the final vaping compound or e-liquid.

The of herbal powder extract of the instant invention can be selected from any of or combination of the following:
1) Kanna (sceletium tortuosum),
2) Salvia (Salvia divinorum),
3) Salvia eivinorm,
4) Kratom (Mitragyna speciosa),
5) Celandine poppy (Stylophorum diphyllum),
6) Mugwort (Artemisia),
7) Coltsfoot leaf (Tussilago farfara),
8) California poppy (Eschscholzia californica),
9) Sinicuichi (Heimia salicifolia),
10) St. John's Wort (Hypericum perforatum),
11) Yerba lenna yesca (Artemisia scoparia),
12) Calea zacatechichi (Calea ternifolia),
13) Blue lotus (Nymphaea caerulea),
14) Leonurus sibericus (Leonurus sibiricus),
15) Wild dagga (Leonotis leonurus),
16) Valarien (Valeriana officinalis),
17) Klip dagga (Leonotis nepetifolia),
18) Damiana (Turnera diffusa),
19) Kava (Piper methysticum),
20) Scotch broom tops (Cytisus scoparius),
21) Indian warrior (Pedicularis densiflora),
22) Wild lettuce (Lactuca virosa),
23) Skullcap (Scutellaria lateriflora),
24) Red Clover (Trifolium pretense),
25) Psilocybin Mushroom (Psilocybin Cubensis),
26) Psilocin Mushroom (Copelandia),
27) Lions Mane (Hericium Erinaceus),
28) Cordyceps (Cordyceps Sinesis),
29) Reishi Mushroom (Ganoderma Lingzhi),
30) Chaga Mushroom (Inonotus Obliquus),
31) Cacao (Forastero, Criollo, Trinitario),
32) Mimosa shrub (mimosa hostilis),
33) Pluteus,
34)Psilocybe),
35)Gymnopilus, Inocybe, Panaeolus, Pholiotina,
36) Hemp/cannabis (Tetrahydrocannabinol (THC), Cannabidiol (CBD))

The instant invention also envisions the process where a synthetic active ingredient is used in place of a natural extract.

The vaporizing base is a carrier such as vegetable glycerin, propylene glycol, or MCT oil, terpenes and flavoring.

MCT oil is an oil made from a fat called medium-chain triglycerides.

Vegetable glycerin (VG) is a sugar alcohol made from plant oils and it is sweet, odorless viscous liquid.

Propylene glycol (IUPAC name: propane-1,2-diol) is a viscous, colorless liquid, which is nearly odorless but possesses a faintly sweet taste. Its chemical formula is CH3CH(OH)CH2OH. Containing two alcohol groups, it is classed as a diol which is an alcohol containing two hydroxyl groups in its molecule. It is miscible with a broad range of solvents, including water, acetone, and chloroform. In general, glycols are non-irritating and have very low volatility.

Terpenes (carrier/flavoring) are natural aromas and flavors which manufacturers extract from various plants. Scientists estimate that there are approximately 20,000 different terpenes existing with over 100 of them coming from hemp. The instant invention can utilize any food grade terpenes.

Terpenes are ideal for adding flavor to vaporizing base and the possess beneficial properties. Terpenes can bind to receptors in the brain and body to stimulate effects. This essentially is the science of aromatherapy. While nearly all plants contain terpenes, some of the more common sources people encounter them include: cannabis, aromatic herbs, like sage and thyme and citrus fruits.

When you add terpenes to a vaporizing base, they help to supply the body with the “entourage effect.” This is when the herbal extracts and terpenes work “synergistically” to achieve an effect greater than any single ingredient would provide.

Traditionally manufacturers have used terpenes to add natural flavor back into a product. Additionally, manufacturers can also substitute terpenes as the carrier in the vaporizing base instead of using vegetable glycerin, propylene glycol, or MCT oil.

The addition of flavorings makes the vaping compound more desirable for the user because the flavor stimulates the sense of taste. The flavors can be sweet or minty flavor. There are estimated to be over 7,000 different flavors that can be incorporated into the vaping compound. Some of these are natural flavors and some are artificial.

The flavorings used in vaping compound are non-oil based, food-grade flavorings. This means the FDA approved the flavor for consumption. These flavorings come from either a natural source (food or plant) or are artificially synthesized.

The following flavorings can be used with the instant invention and they include but are not limited to:
1) Tobacco flavors such as fire cured, bright leaf, burley, shade, perique, latakia, oriental.
2) Acetyl Pyrazine.
3) Almond,
4) Apple Candy,
5) Apple Jacks Type,
6) Apple Pie,
7) Apricot, Banana, Banana
8) Cream, Banana Split,
9) Bananas Foster,
10) Blueberry,
11) Black Cherry,
12) Black Currant,
13) Black Honey,
14) Blackberry,
15) Blood Orange,
16) Blue Raspberry,
17) Brown sugar,
18) Toffee, Butter cream,
19) Cappuccino,
20) Caramel,
21) Cherry,
22) Cherry Cola,
23) Chocolate,
24) Cinnamon,
25) Citrus,
26) Cocoa,
27) Coconut,
28) Coffee, Apple,
29) Peanut Butter,
30) Pineapple,
31) Grape Juice,
32) Grapefruit,
33) Green Apple,
34) Hibiscus,
35) Honeydew Melon,
36) Huckleberry,
37) Rum,
38) Lemon,
39) Kiwi, Lime,
40) Mandarin Orange,
41) Maple,
42) Passion Fruit,
43) Papaya, Peach,
44) Raspberry,
45) Root Beer,
46) Strawberry,
47) Tangerine,
48) Tea,
49) Vanilla,
50) Watermelon,
51) wild cherry and
52) peach.

Using a Kanna extraction as an example of a typical the extraction process for the active ingredient one can form a Kanna isopropyl extract which can be incorporated into the instant invention by using the following recipe.
1) Add 99% 500 ml of isopropyl alcohol to a 250 ml glass jar having an air tight lid.
2) Add 25 mg of citric acid to the glass jar.
3) Add 80 grams of Kanna which has been finely ground into a powder and place it into the glass jar.
4) Using an ultrasonic mixing device such as Branson Sonifier 450 place the sonicator tip of the Branson Sonifier 450 so that it extends to approximately 2 mm from the bottom of the jar. Set the power supply for controls accordingly to 20% Duty Cycle, Output control to 6 and timer to 8. Cover the beaker with a piece of film to prevent splatter or contamination and sonicate for 5 minutes or until the Kanna is completely suspended in the isopropyl alcohol. Ensure that none of the powder is resting on the bottom of the jar prior to turning off the Branson Sonifier 450. The process can utilize any suitable glass container with an air tight lid.
5) Put the air tight lid over the container and insure it is properly sealed.
6) Store the glass jar containing the mixture at 80 degrees Fahrenheit.
7) Every 8 hours place the glass jar in a ultrasonic bath for 10 minutes to mix the mixture. A ultrasonic bath such as an Branson B-12 Ultrasonic Cleaner provides suitable energy into the mixture to keep the powder suspended in the mixture. Return the jar to storage.
8) After 1 week filter out the plant material into a Pyrex dish using Buchner funnel and filter paper with a coarse porosity (90-130 microns).
9) Squeeze filter paper until all the liquid has been removed from the filter paper. Discard the filter paper and the associated plant material.
10) The next step is the evaporation of the isopropyl alcohol, citric acid and Kanna mixture. Place the Pyrex dish and mixture in an oven or dehydrator set 150 to 175 degrees Fahrenheit. Evaporate the mixture until it is dry.
11) Scrape up the residue from the Pyrex dish and grind into powder. If you so wish you can perform an acetone wash on the residue.
12) The Kanna extract is ready for use in the vaping compound or e-liquid.

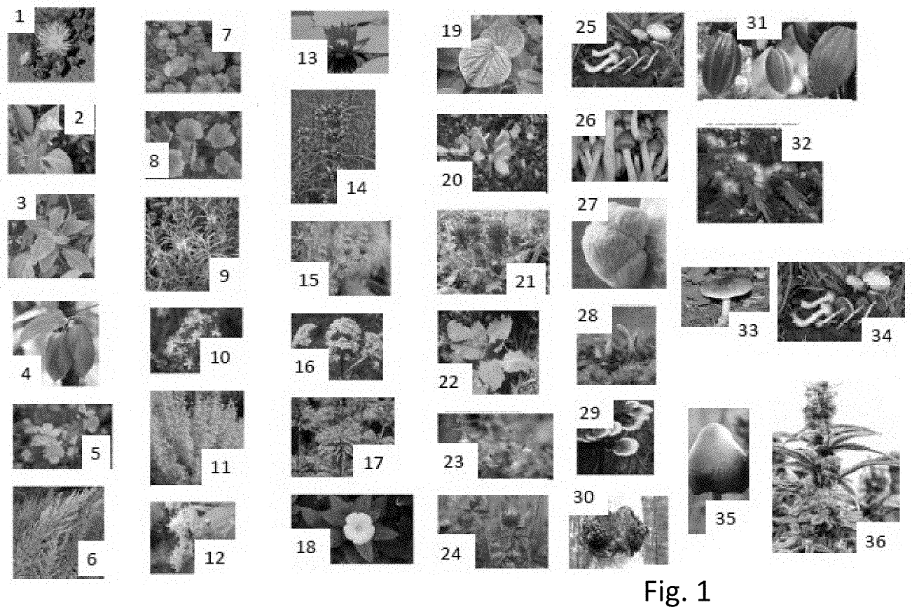

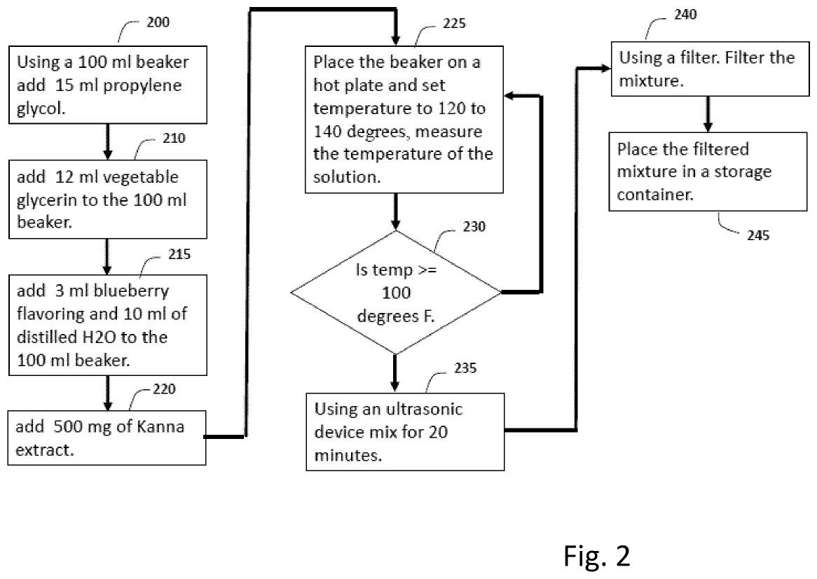

Referring now to the drawings FIG. 1-2, and more particularly to FIG. 1, there is shown a chart of the various plants from which the herbal additives of the present invention are selected from the group.
1) Kanna (sceletium tortuosum),
2) Salvia (Salvia divinorum),
3) Salvia eivinorm,
4) Kratom (Mitragyna speciosa),
5) Celandine poppy (Stylophorum diphyllum),
6) Mugwort (Artemisia),
7) Coltsfoot leaf (Tussilago farfara),
8) California poppy (Eschscholzia californica),
9) Sinicuichi (Heimia salicifolia),
10) St. John's Wort (Hypericum perforatum),
11) Yerba lenna yesca (Artemisia scoparia),
12) Calea zacatechichi (Calea ternifolia),
13) Blue lotus (Nymphaea caerulea),
14) Leonurus sibericus (Leonurus sibiricus),
15) Wild dagga (Leonotis leonurus),
16) Valarien (Valeriana officinalis),
17) Klip dagga (Leonotis nepetifolia),
18) Damiana (Turnera diffusa),
19) Kava (Piper methysticum),
20) Scotch broom tops (Cytisus scoparius),
21) Indian warrior (Pedicularis densiflora),
22) Wild lettuce (Lactuca virosa),
23) Skullcap (Scutellaria lateriflora),
24) Red Clover (Trifolium pretense),
25) Psilocybin Mushroom (Psilocybin Cubensis),
26) Psilocin Mushroom (Copelandia),
27) Lions Mane (Hericium Erinaceus),
28) Cordyceps (Cordyceps Sinesis),
29) Reishi Mushroom (Ganoderma Lingzhi),
30) Chaga Mushroom (Inonotus Obliquus),
31) Cacao (Forastero, Criollo, Trinitario),
32) Mimosa shrub (mimosa hostilis),
33) Pluteus,
34) Psilocybe),
35) Gymnopilus, Inocybe, Panaeolus, Pholiotina,
36) Hemp/Cannabis (Tetrahydrocannabinol (THC), Cannabinol (CBN), Cannabigerol (CBG), Cannabidiol (CBD), etc)

As noted earlier the instant invention anticipates the use of synthetic active ingredients such as citric acid and other synthetic compounds.

The active ingredients can be combined to form a number of beneficial compounds such as adding hemp/cannabis and cacao together into one formulation. Another example would be combining Lions Mane, Cordyceps and hemp (CBD oil) together into one formulation. Any suitable combination of active ingredients is possible depending on the combination of properties desired by the user.

Referring to FIG. 2, there is shown a flow chart of the process. This example uses Kanna extract as the active ingredient and blueberry as the flavor. However, any of the active ingredients or any flavoring desired that is safe for human inhalation can be used. Starting at step 200 Using a 100 ml beaker add 15 ml propylene glycol. Then as described in step 210 add 12 ml vegetable glycerin to the 100 ml beaker. At step 215 add 3 ml blueberry flavoring and 10 ml of distilled water to the 100 ml beaker. The water is used to cut the propylene glycol and vegetable glycerin and is mixed into the solution by the high energy mixing of step 235. The reason for the addition of water to the mixture is that water does not produce a thick cloud of smoke as does the propylene glycol and vegetable glycerin and therefore limits any undesirable feedback from the vapor produced. The percentage of the water, propylene glycol and vegetable glycerin can be varied by the user so they can create a solution with different vapor producing properties. The instant invention envisions vaping compounds or e-liquid comprised of a water content of 1 to 38% by volume percent, propylene glycol content of 20-92 percent by volume, vegetable glycerin content can be from 5 to 85 percent by volume. In an alternative embodiment the user could incorporate MCT oil and the MCT oil content can be from 0 to 40% by volume depending on the vapor qualities desired by the user.

The classical method to create a stable emulsion is to use an emulsifier such as a surfactant that stabilizes emulsions. The surfactant emulsifiers coat droplets within an emulsion and prevent them from coming together, or coalescing. A detergent is a typical surfactant that is used when processing emulsification liquids. However, the use of detergents in a vaping compound is not recommended because detergents are not normally utilized in food and drug applications and therefore a different method is required to overcome the separation tendencies of the oil and water components of the vaping compound. The instant invention utilizes a high shear mixing process which creates a stable emulsion wherein the oil droplets are forced into an emulsion and prevent them from coming together, or coalescing. The use of high shear method to produce a stable emulsion requires introduction of significant energy into the mixture to obtain the finest possible droplet size. The more shear energy introduced into the mixture, the smaller the suspended droplets will become, creating a fine stable emulsion. The high shear mixing of an ultrasonic mixing device such as the Branson Sonifier 450 is ideally suited to this application and can easily achieve emulsions with a droplet size of 2 to 5 microns. Finer emulsions down to 0.5 microns can be obtained by mixing for longer periods of time.

As noted, the amount of water added to the mixture can be from 1 to 38% by volume and depends on the amount of emulsification desired.

At step 220 add the active ingredient which is in this case is 500 mg of Kanna extract. The percentage of active ingredient added to the mixture can vary depending on the desired vaping compound from 0.1 to 50% by weight. This forms the mixture of Kanna, flavor, water, vegetable glycerin and propylene glycol. Then as described in step 225 Place the beaker on a hot plate and set the temperature to 120 to 140 degrees. Measure the temperature of the solution as shown in step 230 and once it reaches 100 degrees Fahrenheit remove the beaker from the hot plate. Using an ultrasonic device mix the solution for 20 minutes as described in step 235. A typical ultrasonic device that can be used is a Branson Sonifier 450. First place the sonicator tip of the Branson Sonifier 450 so that it extends to approximately 2 mm from the bottom of the beaker. Set the power supply for controls accordingly to 20% Duty Cycle, Output control to 6 and timer to 8. Cover the beaker with a piece of film to prevent splatter or contamination and sonicate for 20 minutes. Ensure that all the water and oil has formed an emulsion. Then using a Buchner funnel and a coarse porosity filter paper (90-130 microns) filter the solution as described in step 240. Squeeze filter paper until all the liquid has been removed from the filter paper. Discard the filter paper and any associated material remaining in the filter.

Place the filtered mixture in a storage container as described in step 245 The resulting filtered solution will have an active ingredient content in the range of 0.001 g-5.0 g per mL of solution. To increase the amount of active ingredient the user only has to increase the amount of extract used in the formulation.

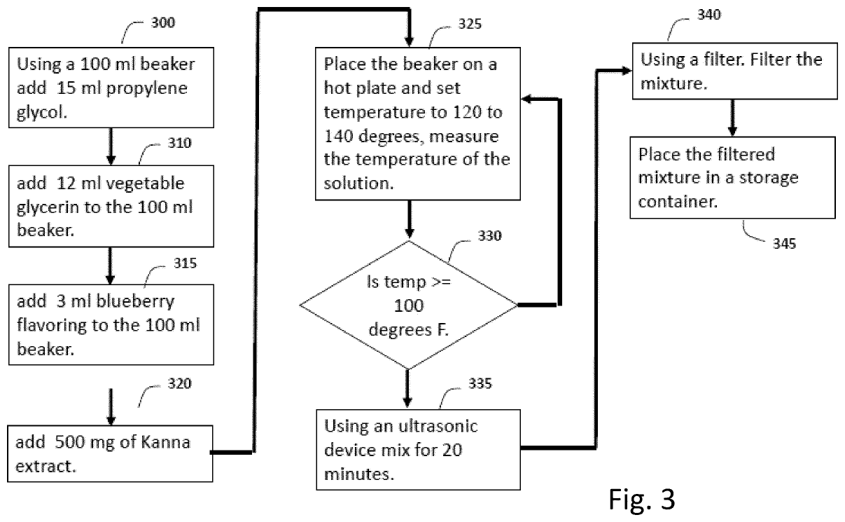

Referring to FIG. 3, there is shown an alternative flow chart of the process. This process does not incorporate a water component and therefor produces more vapor than the previous formulation. This example uses Kanna extract as the active ingredient and blueberry as the flavor. However, any of the active ingredients or any flavoring desired that is safe for human inhalation can be used. Starting at step 300 using a 100 ml beaker add 15 ml propylene glycol. Then as described in step 310 add 12 ml vegetable glycerin to the 100 ml beaker. The percentage of the propylene glycol and vegetable glycerin can be varied by the user so they can create a solution with different vapor producing properties. The instant invention envisions vaping compounds or e-liquid comprised of propylene glycol content of 20-92 percent by volume. The vegetable glycerin content can be from 5 to 85 percent by volume and in an alternative embodiment the MCT oil content can be from 0 to 40% by volume depending on the desired vapor qualities desired by the user. The flavoring content between 0.25-10 percent by volume.

At step 315 add 3 ml blueberry flavoring to the 100 ml beaker. Then as described in step 320 add the active ingredient which is in this case is 500 mg of Kanna extract. The percentage of active ingredient can vary depending on the desired vaping compound from 0.1 to 50% by weight. This forms the mixture of Kanna, flavor, vegetable and propylene glycol. Then as described in step 325 Place the beaker on a hot plate and set the temperature to 120 to 140 degrees. Measure the temperature of the solution as shown in step 330 and once it reaches 100 degrees Fahrenheit remove the beaker from the hot plate. Using an ultrasonic device mix the solution for 20 minutes as described in step 335. A typical ultrasonic device that can be used is a Branson Sonifier 450, place the sonicator tip of the Branson Sonifier 450 so that it extends to approximately 2 mm from the bottom of the beaker. Set the power supply for controls accordingly to 20% Duty Cycle, Output control to 6 and timer to 8. Cover the beaker with a piece of film to prevent splatter or contamination and sonicate for 20 minutes. Then using a Buchner funnel and a coarse porosity filter paper (90-130 microns) filter the solution as described in step 340. Squeeze filter paper until all the liquid has been removed from the filter paper. Discard the filter paper and any associated material remaining in the filter.

Place the filtered mixture in a storage container as described in step 345 The resulting filtered solution will have an active ingredient content in the range of 0.001 g-5.0 g per mL of solution. To increase the amount of active ingredient the user only has to increase the amount of extract used in the formulation.

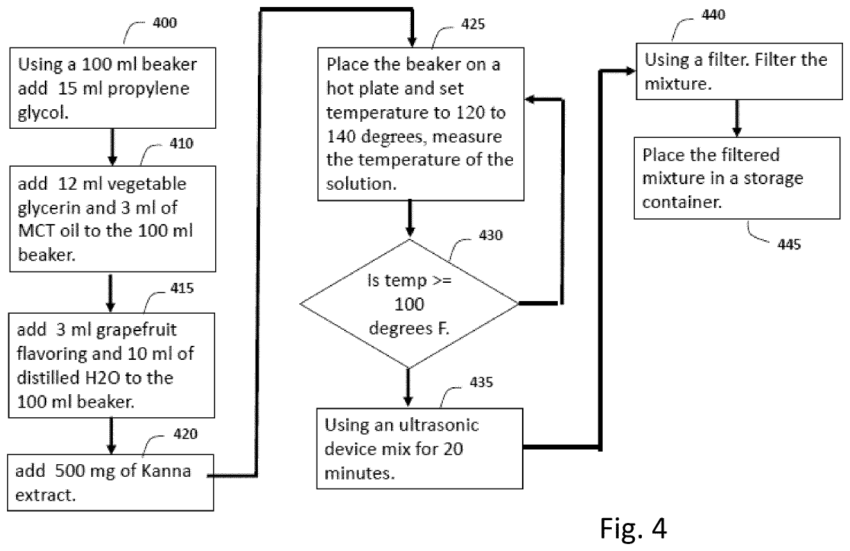

Referring to FIG. 4, there is shown a flow chart of the process. This example uses Kanna extract as the active ingredient and grapefruit as the flavor. However, any of the active ingredients or any flavoring desired that is safe for human inhalation can be used. Starting at step 400 using a 100 ml beaker add 12 ml propylene glycol. Then as described in step 410 add 12 ml vegetable glycerin and 3 ml of MCT oil to the 100 ml beaker. At step 415 add 3 ml grapefruit flavoring which is a terpene and 10 ml of distilled water to the 100 ml beaker. The water is used to cut the propylene glycol and vegetable glycerin and is mixed into the solution by the high energy mixing of step 435. The water does not produce a thick cloud of smoke as does the propylene glycol and vegetable glycerin and therefore limits any undesirable feedback from the vapor produced. The percentage of the water, propylene glycol and vegetable glycerin can vary to create a solution with different vapor producing properties. The instant invention envisions vaping compounds or e-liquid comprised of water content of 1 to 38% by volume percent, propylene glycol content of 20-92 percent by volume. The vegetable glycerin content can be from 5 to 85 percent by volume and in an alternative embodiment the MCT oil content can be from 0 to 40% by volume depending on the desired vapor qualities desired by the user. The terpene content of 1-50 percent by volume and the flavoring content between 0.25-10 percent by volume.

The classical method to create a stable emulsion is to use an emulsifier such as a surfactant that stabilizes emulsions. The surfactant emulsifiers coat droplets within an emulsion and prevent them from coming together, or coalescing. A detergent is a surfactant that has cleaning properties in dilute solutions. However, the use of detergents in a vaping compound is not recommended because detergents are not normally utilized in food and drug applications and a different method is required to overcome the separation of the oil and water components of the vaping compound. The instant intention utilizes a high shear mixing process which creates a stable emulsion wherein the oil droplets are forced into an emulsion and prevent them from coming together, or coalescing. The use of high shear method to produce a stable emulsion requires the input of energy into the mixture to obtain the finest possible droplet size. The more shear energy introduced into the mixture, the smaller the suspended droplets will become, creating a fine stable emulsion. The high shear mixing of an ultrasonic disintegrator is ideally suited to this application and can easily achieve emulsions with a droplet size of 2 to 5 microns. Finer emulsions down to 0.5 microns can be obtained by mixing for longer periods of time.

As noted, the amount of water added to the mixture can be from 1 to 38% by volume and depends on the amount of emulsification desired.

At step 420 add the active ingredient which is in this case 500 mg of Kanna extract. The percentage of active ingredient can vary depending on the desired vaping compound from 0.1 to 50% by weight of the vaping compound or e-liquid. This forms the mixture of Kanna, flavor, vegetable and propylene glycol. Then as described in step 425 place the beaker on a hot plate and set the temperature to 120 to 140 degrees. Measure the temperature of the solution as shown in step 430 and once it reaches 100 degrees Fahrenheit remove the beaker from the hot plate. Using an ultrasonic device mix the solution for 20 minutes as described in step 435. A typical ultrasonic device that can be used is a Branson Sonifier 450, place the sonicator tip of the Branson Sonifier 450 so that it extends to approximately 2 mm from the bottom of the beaker. Set the power supply for controls accordingly to 20% Duty Cycle, Output control to 6 and timer to 8. Cover the beaker with a piece of film to prevent splatter or contamination and sonicate for 20 minutes. Then using a Buchner funnel and a coarse porosity filter paper (90-130 microns) filter the solution as described in step 440. Squeeze filter paper until all the liquid has been removed from the filter paper. Discard the filter paper and any associated material remaining in the filter.

Place the filtered mixture in a storage container as described in step 445 The resulting filtered solution will have an active ingredient content in the range of 0.001 g-5.0 g per mL of solution. To increase the amount of active ingredient the user only has to increase the amount of extract used in the formulation.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A vaping solution comprised of the following ingredients:
a. a food grade oil;
b. water;
c. food grade flavoring; and
d. active ingredients.

2. The vaping solution of claim 1 wherein said food grade oil is selected from the group consisting of propylene glycol, MCT oil and vegetable glycerin.

3. The vaping solution of claim 1 wherein said food grade oil comprises of a mixture of propylene glycol, MCT oil and vegetable glycerin.

4. The vaping solution of claim 1 wherein said food grade oil comprises of a mixture of propylene glycol, and MCT oil.

5. The vaping solution of claim 1 wherein said food grade oil comprises of a mixture of propylene glycol, and vegetable glycerin.

6. The vaping solution of claim 1 wherein said food grade oil comprises of a mixture of MCT oil and vegetable glycerin.

7. The vaping solution of claim 1 wherein said food grade flavoring is selected from the group consisting of tobacco flavors such as fire cured, bright leaf, burley, shade, perique, latakia, oriental, acetyl pyrazine, almond, apple candy, apple jacks, apple pie, apricot, banana, banana cream, banana split, bananas foster, blueberry, back cherry, black currant, black honey, blackberry, blood orange, blue raspberry, brown sugar, toffee, butter cream, cappuccino, caramel, cherry, cherry cola, chocolate, cinnamon, citrus, cocoa, coconut, coffee, apple, peanut butter, pineapple, grape juice, grapefruit, green apple, hibiscus, honeydew melon, huckleberry, rum, lemon, kiwi, lime, mandarin orange, maple, passion fruit, papaya, peach, raspberry, root beer, strawberry, tangerine, tea, vanilla, watermelon, wild cherry and peach.

8. The vaping solution of claim 1 wherein said active ingredient is state is selected from the group consisting of powder, liquid, and oleoresin.

9. The vaping solution of claim 1 wherein said active ingredients is selected from the group consisting of:
a. Kanna (sceletium tortuosum),
b. Salvia (Salvia divinorum),
c. Salvia eivinorm,
d. Kratom (Mitragyna speciosa),
e. Celandine poppy (Stylophorum diphyllum),
f. Mugwort (Artemisia),
g. Coltsfoot leaf (Tussilago farfara),
h. California poppy (Eschscholzia californica),
i. Sinicuichi (Heimia salicifolia),
j. St. John's Wort (Hypericum perforatum),
k. Yerba lenna yesca (Artemisia scoparia),
l. Calea zacatechichi (Calea ternifolia),
m. Blue lotus (Nymphaea caerulea),
n. Leonurus sibericus (Leonurus sibiricus),
o. Wild dagga (Leonotis leonurus),
p. Valarien (Valeriana officinalis),
q. Klip dagga (Leonotis nepetifolia),
r. Damiana (Turnera diffusa),
s. Kava (Piper methysticum),
t. Scotch broom tops (Cytisus scoparius),
u. Indian warrior (Pedicularis densiflora),
v. Wild lettuce (Lactuca virosa),
w. Skullcap (Scutellaria lateriflora),
x. Red Clover (Trifolium pretense),
y. Psilocybin Mushroom (Psilocybin Cubensis),
z. Psilocin Mushroom (Copelandia),
aa. Lions Mane (Hericium Erinaceus),
bb. Cordyceps (Cordyceps Sinesis),
cc. Reishi Mushroom (Ganoderma Lingzhi),
dd. Chaga Mushroom (Inonotus Obliquus),
ee. Cacao (Forastero, Criollo, Trinitario),
ff. Mimosa shrub (mimosa hostilis),
gg. Pluteus,
hh. Psilocybe),
ii. Gymnopilus, Inocybe, Panaeolus, Pholiotina,
jj. Hemp/cannabis (Tetrahydrocannabinol (THC), and Cannabidiol (CBD)).

10. The vaping solution of claim 1 wherein the percentage of said water in the vaping solution is from 1-38 percent by volume.

11. The vaping solution of claim 2 wherein the percentage of said propylene glycol in the vaping solution is from 20-92 percent by volume.

12. The vaping solution of claim 2 wherein the percentage of said vegetable glycerin content can be from 5 to 85 percent by volume

13. The vaping solution of claim 2 wherein the percentage of said MCT oil content can be from 0 to 40% by volume.

14. The vaping solution of claim 1 wherein the percentage of said active ingredient content can be from 0.1 to 50% by volume.

15. A method of creating a vaping solution of claim 1 wherein:
a. 27 ml of food grade oil is placed in a beaker;
b. 3 ml of food grade flavoring and 10 ml of water are placed in said beaker;
c. dry active ingredients is placed in said beaker; and
d. the contents of said beaker is mixed with an ultrasonic mixing device

16. The method of creating vaping solution of claim 15 wherein said food grade oil is selected from the group consisting of propylene glycol, MCT oil and vegetable glycerin.

17. The method of creating vaping solution of claim 15 wherein said food grade oil comprises of a mixture of propylene glycol, MCT oil and vegetable glycerin.

18. The method of creating vaping solution of claim 15 wherein said food grade oil comprises of a mixture of propylene glycol, and MCT oil.

19. The method of creating vaping solution of claim 15 wherein said food grade oil comprises of a mixture of propylene glycol, and vegetable glycerin.

20. The method of creating vaping solution of claim 15 wherein the percentage of said active ingredient content can be from 0.1 to 50% by volume.

21. The method of creating vaping solution of claim 15 wherein said food grade flavoring is selected from the group consisting of Tobacco flavors such as fire cured, bright leaf, burley, shade, perique, latakia, oriental, acetyl pyrazine, almond, apple candy, apple jacks, apple pie, apricot, banana, banana cream, banana split, bananas foster, blueberry, back cherry, black currant, black honey, blackberry, blood orange, blue raspberry, brown sugar, toffee, butter cream, cappuccino, caramel, cherry, cherry cola, chocolate, cinnamon, citrus, cocoa, coconut, coffee, apple, peanut butter, pineapple, grape juice, grapefruit, green apple, hibiscus, honeydew melon, huckleberry, rum, lemon, kiwi, lime, mandarin orange, maple, passion fruit, papaya, peach, raspberry, root beer, strawberry, tangerine, tea, vanilla, watermelon, wild cherry and peach.

22. The method of creating vaping solution of claim 15 wherein said dry active ingredients is selected from the group consisting of:
a. Kanna (sceletium tortuosum),
b. Salvia (Salvia divinorum),
c. Salvia eivinorm,
d. Kratom (Mitragyna speciosa),
e. Celandine poppy (Stylophorum diphyllum),
f. Mugwort (Artemisia),
g. Coltsfoot leaf (Tussilago farfara),
h. California poppy (Eschscholzia californica),
i. Sinicuichi (Heimia salicifolia),
j. St. John's Wort (Hypericum perforatum),
k. Yerba lenna yesca (Artemisia scoparia),
l. Calea zacatechichi (Calea ternifolia),
m. Blue lotus (Nymphaea caerulea),
n. Leonurus sibericus (Leonurus sibiricus),
o. Wild dagga (Leonotis leonurus),
p. Valarien (Valeriana officinalis),
q. Klip dagga (Leonotis nepetifolia),
r. Damiana (Turnera diffusa),
s. Kava (Piper methysticum),
t. Scotch broom tops (Cytisus scoparius),
u. Indian warrior (Pedicularis densiflora),
v. Wild lettuce (Lactuca virosa),
w. Skullcap (Scutellaria lateriflora),
x. Red Clover (Trifolium pretense),
y. Psilocybin Mushroom (Psilocybin Cubensis),
z. Psilocin Mushroom (Copelandia),
aa. Lions Mane (Hericium Erinaceus),
bb. Cordyceps (Cordyceps Sinesis),
cc. Reishi Mushroom (Ganoderma Lingzhi),
dd. Chaga Mushroom (Inonotus Obliquus),
ee. Cacao (Forastero, Criollo, Trinitario),
ff. Mimosa shrub (mimosa hostilis),
gg. Pluteus,
hh. Psilocybe),
ii. Gymnopilus, Inocybe, Panaeolus, Pholiotina,
jj. ‘Hemp/cannabis (Tetrahydrocannabinol (THC), and Cannabidiol (CBD)).
